(19) United States
(12) Patent Application Publication
(10) Pub. No.: US 2025/0019617 A1
(43) Pub. Date: Jan. 16, 2025

(54) COMPOSITIONS AND METHODS

(71) Applicant: THE SUSTAINABLE CARE COMPANY LIMITED, Auckland (NZ)

(72) Inventors: Mark Christian Sorensen, Auckland (NZ); David Jonathan Hassell, Auckland (NZ); Bernard Cecil Kimble, Auckland (NZ); Dennis Lampasa Macalintal, Auckland (NZ)

(21) Appl. No.: 18/714,679

(22) PCT Filed: Nov. 30, 2022
(86) PCT No.: PCT/NZ2022/050160

§ 371 Date: May 30, 2024

(30) Foreign Application Priority Data
Nov. 30, 2021 (NZ) NZ782121

(51) Int. Cl. C11D 3/22; A61K 8/73; A61Q 5/02; A61Q 19/10; C11D 1/83; C11D 11/00; C11D 11/02; C11D 17/00

(52) U.S. Cl. CPC C11D 3/222; A61K 8/73; A61Q 5/02; A61Q 19/10; C11D 1/83; C11D 11/0094; C11D 11/02; C11D 17/0047; A61K 2800/48; A61K 2800/5422; A61K 2800/5424

(57) ABSTRACT

This disclosure relates to a solid concentrate composition and/or a method of preparing a liquid cleaning composition from a solid concentrate composition and/or a method of cleaning a surface and/or a method of preparing a solid concentrate composition. More particularly, this disclosure relates to a solid concentrate composition comprising a fast-acting thickening agent, a slow-acting thickening agent and a surfactant.

### TECHNICAL FIELD

This disclosure relates to a solid concentrate composition and/or a method of preparing a liquid cleaning composition from a solid concentrate composition and/or a method of cleaning a surface and/or a method of preparing a solid concentrate composition. More particularly, this disclosure relates to a solid concentrate composition comprising a fast-acting thickening agent, a slow-acting thickening agent and a surfactant.

### BACKGROUND ART

Increasing awareness of the ecological impact of consumer products has driven demand for more environmentally friendly products. Such products include concentrated cleaning products (for example, that include less fillers) that can be used in smaller quantity that previous products, or concentrated products that may be diluted by the consumer to make up to normal quantities. Delivering cleaning products in a concentrated form reduces waste from packaging and reduces the footprint from storage and transport of the products.

Concentrated products that are to be diluted by the consumer before use are usually provided in a liquid form that only requires simple dilution with water and mixing to achieve a useable product. More recently, solid concentrated products, such as concentrated tablets and powders, have been developed that can be diluted with water in refill dispensing bottles by the consumer to make liquid cleaning products. Solid concentrated products have the added advantages of allowing the use of more environmentally friendly packaging materials as well as further reducing the footprint from storage and transport of the products. However, a limitation of solid concentrated products is formulating a concentrated product that may be readily diluted by home consumers to provide a liquid cleaning composition that has the desired properties, such as viscosity.

Viscous cleaning products such as shampoos and dishwashing liquids are traditionally produced in large mixing vessels fitted with powerful mixing equipment capable of dispersing thickening agents and ensuring that the finished product is homogeneous; without lumps or occlusions. Mixing a powdered concentrate with cold water in a domestic sized bottle poses problems mainly due to the lack of energy required to adequately disperse the thickening agents without creating lumps or “fisheyes” of dry powder coated with a gelatinous skin.

One solution is to use a slow hydrating gelling agent that can be effectively wet out before starting to thicken. The problem with this approach is that the gelling agent tends to sink to the bottom of the container. When the gelling agent does eventually hydrate fully, it results in a high viscosity layer on the bottom and a lower viscosity layer on the top that are difficult to redisperse. Further, the consumer has to wait a long time before using the product.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### SUMMARY

It is an object of this disclosure to provide a solid concentrate composition and/or a method of preparing a liquid cleaning composition and/or a liquid cleaning composition and/or a method of cleaning a surface and/or a method of preparing a solid concentrate composition which goes at least some way towards overcoming one or more of the abovementioned problems or difficulties, and/or to at least provide the public with a useful choice.

In a first aspect the present invention provides a solid concentrate composition comprising: a fast-acting thickening agent, a slow-acting thickening agent, and a surfactant.

In a second aspect the invention provides a method of preparing a liquid cleaning composition, the method comprising:
a) providing a solid concentrate composition comprising:
   (i) a fast-acting thickening agent,
   (ii) a slow-acting thickening agent, and
   (iii) a surfactant; and
b) adding the solid concentrate composition to water, or adding water to the solid concentrate composition, to provide the liquid cleaning composition.

In a third aspect the invention provides a liquid cleaning composition prepared according to the method of the second aspect.

In a fourth aspect the invention provides a method of cleaning a surface, the method comprising:
a) providing a solid concentrate composition comprising:
   (i) a fast-acting thickening agent,
   (ii) a slow-acting thickening agent, and
   (iii) a surfactant; and
b) adding the solid concentrate composition to water, or adding water to the solid concentrate composition, to provide a liquid cleaning composition; and
c) applying the liquid cleaning composition to the surface.

In a fifth aspect the invention provides a method of preparing a solid concentrate composition, the method comprising combining:
a) a fast-acting thickening agent,
b) a slow-acting thickening agent, and
c) a surfactant.

Any of the aforementioned features or embodiments or aspects may be combined with one or more of the other features or embodiments or aspects as described herein.

In some embodiments, the solid concentrate composition comprises about 0.1 to 5.0% w/w of the fast-acting thickening agent. In some embodiments, the solid concentrate composition comprises about 0.1 to 4% w/w of the fast-acting thickening agent. In some embodiments, the solid concentrate composition comprises about 0.1 to 3% w/w of the fast-acting thickening agent. In some embodiments, the solid concentrate composition comprises about 0.1 to 2% w/w of the fast-acting thickening agent. In some embodiments, the solid concentrate composition comprises about 0.2 to 2% w/w of the fast-acting thickening agent. In some embodiments, the solid concentrate composition comprises about 0.3 to 2% w/w of the fast-acting thickening agent. In some embodiments, the solid concentrate composition comprises about 0.3 to 1% w/w of the fast-acting thickening agent.

In some embodiments, the solid concentrate composition comprises, less than about 5% w/w, less than about 4% w/w, less than about 3% w/w, less than about 2% w/w, or less than about 1% w/w of the fast-acting thickening agent.

In some embodiments, the solid concentrate composition comprises about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 2.0% w/w, about 2.5% w/w, about 3.0% w/w, about 3.5% w/w, about 4.0% w/w, about 4.5% w/w, or about 5.0% w/w of the fast-acting thickening agent.

In some embodiments, the solid concentrate composition comprises about 0.5% w/w of the fast-acting thickening agent. In some embodiments, the solid concentrate composition comprises about 1% w/w of the fast-acting thickening agent.

In some embodiments, the fast-acting thickening agent provides at least about 80% of its total thickening action within about 2 minutes of addition to a liquid. In some embodiments the fast-acting thickening agent provides at least about 80% of its total thickening action within about 1 minute of addition to a liquid. In some embodiments, the fast-acting thickening agent provides at least about 90% of its total thickening action within about 2 minutes of addition to a liquid. In some embodiments, the fast-acting thickening agent provides at least about 90% of its total thickening action within about 1 minute of addition to a liquid.

In some embodiments, the liquid is water.

In some embodiments the fast-acting thickening agent is a gelling agent.

In some embodiments, the fast-acting thickening agent is a thixotropic agent.

In some embodiments, the fast-acting thickening agent is selected from the group consisting of xanthan gum, guar gum, locust bean gum, carbomer, natural clays (such as attapulgite clay and montmorillonite clay), synthetic clays (such as Laponite) and a combination of any two or more thereof. In some embodiments, the fast-acting thickening agent is xanthan gum.

In some embodiments, the solid concentrate composition comprises about 1 to 20% w/w of the slow-acting thickening agent. In some embodiments, the solid concentrate composition comprises about 2 to 18% w/w of the slow-acting thickening agent. In some embodiments, the solid concentrate composition comprises about 5 to 16% w/w of the slow-acting thickening agent. In some embodiments, the solid concentrate composition comprises about 8 to 15% w/w of the slow-acting thickening agent.

In some embodiments, the solid concentrate composition comprises about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, or about 20% w/w of the slow-acting thickening agent.

In some embodiments, the composition comprises about 10% w/w of the slow-acting thickening agent. In some embodiments, the composition comprises about 11% w/w of the slow-acting thickening agent.

In some embodiments, the slow-acting thickening agent provides less than about 80% of its total thickening action about 1 minute after addition to a liquid. In some embodiments, the slow-acting thickening agent provides less than 80% of its total thickening action 2 minutes after addition to a liquid.

In some embodiments, the slow-acting thickening agent provides at least about 80% of its total thickening action within about 10 minutes to 3 hours of addition to a liquid. In some embodiments, the slow-acting thickening agent provides at least about 90% of its total thickening action within about 10 minutes to 3 hours of addition to a liquid.

In some embodiments, the liquid is water.

In some embodiments, the slow-acting thickening agent is selected from the group consisting of carboxy methyl cellulose (CMC), hydroxy ethyl cellulose (HEC), methyl hydroxy ethyl cellulose (MHEC), methyl hydroxy propyl cellulose (MHPC), hydroxypropyl methyl cellulose (HPMC), modified starches, carrageenan, gellan and a combination of any two or more thereof. In some embodiments, the slow-acting thickening agent is HEC.

In some embodiments, the slow-acting thickening agent is a less thixotropic agent than the fast-acting thickening agent. In some embodiments, the slow-acting thickening agent is a non-thixotropic agent.

In some embodiments, the non-thixotropic thickening agent is HEC.

In some embodiments, the solid concentrate composition comprises the fast-acting thickening agent and the slow-acting thickening agent in a ratio of about 1:5 to 1:40 by weight.

In some embodiments, the solid concentrate composition comprises the fast-acting thickening agent and the slow-acting thickening agent in a ratio of about 1:5 to 1:30 by weight.

In some embodiments, the solid concentrate composition comprises the fast-acting thickening agent and the slow-acting thickening agent in a ratio of about 1:5 to 1:25 by weight.

In some embodiments, the solid concentrate composition comprises the fast-acting thickening agent and the slow-acting thickening agent in a ratio of about 1:10 to 1:22 by weight.

In some embodiments, the surfactant is selected from the group consisting of an anionic surfactant, an amphoteric surfactant, a non-ionic surfactant and a combination of any two or more thereof. For example, there may be more than one from a single type of surfactant and/or more than one type of surfactant.

In some embodiments, the anionic surfactant is selected from the group consisting of a secondary alkyl sulfonate, an olefin sulfonate, a soap of fatty acids, an alkylbenzene sulfonate, an alcohol sulfate, an alcohol ether sulfate, an alkyl sulfate, an alkyl sulphosuccinate, a sulfosuccinic derivative, an alkyl ether carboxylate, an alkyl phosphate, an alkylether phosphate, a fatty acid isethionate, an acyl sarcosinate, a fatty acid/amino acid ester, a fatty alcohol amino acid ester, a fatty alcohol amino acid ester adduct, an amino acid derivative, a salt thereof and a combination of any two or more thereof.

In some embodiments, the anionic surfactant is a secondary alkyl sulfonate or salt thereof. In some embodiments, the anionic surfactant is a secondary alkyl sulfonate or a sodium or potassium salt thereof.

In some embodiments, the secondary alkyl sulfonate is a C₁₃-C₁₈ secondary alkyl sulfonate, or salt thereof. In some embodiments, the secondary alkyl sulfonate is a C₁₄-C₁₆ secondary alkyl sulfonate, or salt thereof.

In some embodiments, the anionic surfactant is an olefin sulfonate or salt thereof. In some embodiments, the anionic surfactant is an olefin sulfonate or a sodium or potassium salt thereof. In some embodiments, the olefin sulfonate is a long-chain olefin sulfonate or salt thereof. In some embodiments, the olefin sulfonate is an alpha olefin sulfonate or salt thereof. In some embodiments, the olefin sulfonate is a C₁₂-C₁₈ olefin sulfonate, or salt thereof. In some embodiments, the olefin sulfonate is a C₁₄-C₁₆ olefin sulfonate, or salt thereof. In some embodiments, the olefin sulfonate is Hostapur OSB.

In some embodiments, the alkyl sulfate is selected from the group consisting of palm sulfate, coco sulfate, lauryl sulfate, a salt thereof and a combination of any two or more thereof.

In some embodiments, the amino acid derivative is a derivative of an amino acid and a fatty acid.

In some embodiments, the amino acid derivative is a salt of lauroyl glutamate.

In some embodiments, the amino acid derivative is sodium lauroyl glutamate.

In some embodiments, the non-ionic surfactant is selected from the group consisting of an ethoxylated fatty alcohol, an ethoxylated nonyl phenol, an ethoxylated fatty acid, a fatty acid alanolamide, an ethoxylated fatty amine, a fatty amine oxides glyceryl fatty ester, a sorbitan, an ethoxylated sorbitan ester, a sucrose ester, an alkyl polyglucoside, an ethylene/propylene oxide block copolymer, an ethoxylated fatty alcohol, a propoxylated fatty alcohol and a combination of any two or more thereof.

In some embodiments, the amphoteric surfactant is selected from the group consisting of an alkyl dimethyl betaine, an alkyl amido betaine, an alkyl sulphobetaine, an alkyl amido sulphobetaine, an imidazoline betaine derivative, an alkyl amino propionate and a combination of any two or more thereof.

In some embodiments, the surfactant is a sodium or potassium salt.

In some embodiments, the surfactant is a combination comprising one or more anionic surfactant(s) and one or more non-ionic surfactant(s).

In some embodiments, the surfactant is a combination comprising a secondary alkyl sulfonate, a further anionic surfactant and a non-ionic surfactant.

In some embodiments, the surfactant is a combination selected from two or more of a secondary alkyl sulphonate, a C₁₄-C₁₆ alpha olefin sulfonate, a cocosulfate, a lauroyl glutamate, an alkyl polyglucoside, and a salt thereof.

In some embodiments, the surfactant is a combination comprising a secondary alkyl sulphonate, sodium lauroyl glutamate and C₁₀-C₁₄ alkyl polyglucoside. In some embodiments, the surfactant is a combination comprising a C₁₄-C₁₆ alpha olefin sulfonate, sodium lauroyl glutamate and C₁₀-C₁₄ alkyl polyglucoside.

In some embodiments, the surfactant is a combination comprising a secondary alkyl sulphonate and sodium lauroyl glutamate. In some embodiments, the surfactant is a combination comprising a C₁₄-C₁₆ alpha olefin sulfonate and sodium lauroyl glutamate.

In some embodiments, the surfactant is a combination comprising a secondary alkyl sulfonate, sodium cocosulfate, and sodium lauroyl glutamate. In some embodiments, the surfactant is a combination comprising a C₁₄-C₁₆ alpha olefin sulfonate, sodium cocosulfate, and sodium lauroyl glutamate.

In some embodiments, the surfactant is a combination comprising a sodium lauroyl glutamate and C₁₀-C₁₄ alkyl polyglucoside.

In some embodiments, the surfactant is a combination comprising a secondary alkyl sulfonate, sodium lauroyl glutamate and C₁₀-C₁₄ alkyl polyglucoside. In some embodiments, the surfactant is a combination comprising a C₁₄-C₁₆ alpha olefin sulfonate, sodium lauroyl glutamate and C₁₀-C₁₄ alkyl polyglucoside.

In some embodiments the surfactant is not a sulfate.

In some embodiments, the solid concentrate composition or the liquid cleaning composition does not comprise a sulfate-containing surfactant.

In some embodiments, the solid concentrate composition comprises at least about 40% w/w of one or more surfactant(s). In some embodiments, the solid concentrate composition comprises at least about 50% w/w of the surfactant.

In some embodiments, the solid concentrate composition comprises about 30 to 70% w/w of one or more surfactant(s), for example, about 30% w/w, about 35% w/w, about 40% w/w, about 45% w/w, about 55% w/w, about 60% w/w, about 65% w/w or about 70% w/w. In some embodiments, the solid concentrate composition comprises about 40 to 60% w/w of one or more surfactant(s). In some embodiments, the solid concentrate composition comprises about 50 to 55% w/w of one or more surfactant(s).

In some embodiments, the surfactant is a combination comprising about 40 to 60% w/w of an anionic surfactant and about 1 to 10% w/w of a non-ionic surfactant. In some embodiments, the surfactant is a combination comprising about 50% w/w of an anionic surfactant and about 1 to 10% w/w of a non-ionic surfactant.

In some embodiments, the solid concentrate composition comprises one or more of a pH modifying or stabilising agent(s), a chelating agent(s), an inert filler(s) or carrier agent(s), a fragrance(s), a preservative(s), a humectant(s), a conditioning agent(s) and a combination of any two or more thereof.

In some embodiments, the solid concentrate composition comprises one or more pH modifying or stabilising agent(s).

In some embodiments, the pH modifying or stabilising agent(s) comprises one or more acidifying agent(s).

In some embodiments, the pH modifying or stabilising agent(s) comprises one or more acidifying agent(s) and/or one or more conjugate base(s).

In some embodiments, the acidifying agent(s) is selected from the group consisting of citric acid, ascorbic acid, tartaric acid, malic acid, sulfamic acid and a combination of any two or more thereof.

In some embodiments, the solid concentrate composition comprises about 0 to 20% w/w or about 0.1 to 20% w/w of the acidifying agent(s), for example, about 1% w/w, about 5% w/w, about 10% w/w, about 15% w/w or about 20% w/w. In some embodiments, the solid concentrate composition comprises about 1 to 10% w/w of the acidifying agent(s), for example, about 1% w/w, about 2% w/w, about 3% w/w, about 4% w/w, about 5% w/w, about 6% w/w, about 7% w/w, about 8% w/w, about 9% w/w or about 10% w/w. In some embodiments, the solid concentrate composition comprises about 1 to 5% w/w of the acidifying agent(s). In some embodiments, the solid concentrate composition comprises about 1% w/w of the acidifying agent(s). In some embodiments, the solid concentrate composition comprises about 0.5% w/w of the acidifying agent(s).

In some embodiments, the solid concentrate composition comprises one or more chelating agent(s).

In some embodiments, the chelating agent(s) is selected from the group consisting of citric acid, sodium citrate, ethylenediaminetetraacetic acid (EDTA), nitrilotriacetic acid (NTA), glutamic acid diacetic acid (GLDA), methylglycine N,N-diacetic acid (MGDA), phosphates, phosphonates, polyacrylic acids and their salts, polymaleic acid and their salts, polycarboxylates and a combination of any two or more thereof.

In some embodiments, the solid concentrate composition comprises about 0 to 30% w/w or about 0.1 to 30% w/w of the chelating agent(s), for example, about 1% w/w, about 5% w/w, about 10% w/w, about 15% w/w, about 20% w/w, about 25% w/w, or about 30% w/w. In some embodiments, the solid concentrate composition comprises about 1 to 10% w/w of the chelating agent(s), for example, about 1% w/w, about 2% w/w, about 3% w/w, about 4% w/w, about 5% w/w, about 6% w/w, about 7% w/w, about 8% w/w, about 9% w/w or about 10% w/w. In some embodiments, the solid concentrate composition comprises about 2% w/w of the chelating agent(s). In some embodiments, the solid concentrate composition comprises about 4% w/w of the chelating agent(s).

In some embodiments, the solid concentrate composition comprises one or more inert filler(s) or carrier agent(s).

In some embodiments, the inert filler(s) or carrier agent(s) is selected from the group consisting of sodium chloride, potassium chloride, sodium citrate, maltodextrin, sucrose, sorbitol, sodium tripolyphosphate, sodium sulfate, potassium sulfate, magnesium sulfate and a combination of any two or more thereof.

In some embodiments, the solid concentrate composition comprises about 0 to 50% w/w or about 0.1 to 50% w/w of the inert filler(s), for example, about 5 to 45% w/w, about 10 to 40% w/w, about 15 to 35% w/w or about 20 to 30% w/w. In some embodiments, the solid concentrate composition comprises about 20 to 30% w/w of the inert filler(s), for example, about 20% w/w, about 21% w/w, about 22% w/w, about 23% w/w, about 24% w/w, about 25% w/w, about 26% w/w, about 27% w/w, about 28% w/w, about 29% w/w or about 30% w/w. In some embodiments, the solid concentrate composition comprises about 15 to 25% w/w of maltodextrin. In some embodiments, the solid concentrate composition comprises about 10 to 20% w/w of maltodextrin and about 5 to 15% w/w of sodium sulfate.

In some embodiments, the solid concentrate composition comprises one or more fragrance(s).

In some embodiments, the fragrance(s) is selected from the group consisting of essential oils, other plant extracts, synthetic fragrance compounds and a combination of any two or more thereof.

In some embodiments, the solid concentrate composition comprises about 0 to 20% w/w or about 0.1 to 20% w/w of the fragrance(s), for example, about 1 to 10% w/w or about 1 to 5% w/w of the fragrance(s). In some embodiments, the solid concentrate composition comprises about 2% w/w.

In some embodiments, the solid concentrate composition comprises one or more preservative(s).

In some embodiments, the preservative(s) is selected from the group consisting of natural extracts or synthetic equivalents, acidic preservatives and/or their salts, phenolic compounds, halogenated compounds, quaternary ammonium compounds and a combination of any two or more thereof.

In some embodiments, the natural extract or synthetic equivalent is selected from the group consisting of hinokitiol, eugenol, tea tree oil, eucalyptus oil and thymol. In some embodiments, the acidic preservative and/or their salt is selected from the group consisting of dehydroacetic acid, benzoic acid, sorbic acid, formic acid, lactic acid, propionic acid and salts thereof.

In some embodiments, the preservative is sodium benzoate.

In some embodiments, the phenolic compound is selected from the group consisting of phenoxyethanol, benzyl alcohol and phenethyl alcohol.

In some embodiments, the solid concentrate composition comprises one or more humectant(s).

In some embodiments, the humectant(s) is selected from the group consisting of betaine, glycerin, sodium lactate, glycols (such as pentylene glycol, propylene glycol and dipropylene glycol), sodium pyroglutamic acid, urea, sorbitol, xylitol and a combination of any two or more thereof.

In some embodiments, the solid concentrate composition comprises about 0 to 20% w/w or about 0.1 to 20% w/w of the humectant(s), for example, about 1% w/w, about 5% w/w, about 10% w/w, about 15% w/w, or about 20% w/w. In some embodiments, the solid concentrate composition comprises about 1 to 10% w/w of the humectant(s), for example, about 1% w/w, about 2% w/w, about 3% w/w, about 4% w/w, about 5% w/w, about 6% w/w, about 7% w/w, about 8% w/w, about 9% w/w or about 10% w/w. In some embodiments, the solid concentrate composition comprises about 5% w/w of the humectant(s).

In some embodiments, the solid concentrate composition comprises one or more conditioning agent(s).

In some embodiments, the conditioning agent(s) is selected from the group consisting of hydroxypropyl guar gum, a polyquaternium (such as polyquaternium 10), lecithin (such as soya or egg lecithin), hydrolysed keratin, vitamin B5, panthenol, behentrimonium chloride and a combination of any two or more thereof.

In some embodiments, the solid concentrate composition comprises about 0 to 30% w/w or about 0.1 to 30% w/w of the conditioning agent(s), for example, about 1% w/w, about 5% w/w, about 10% w/w, about 15% w/w, about 20% w/w, about 25% w/w, or about 30% w/w. In some embodiments, the solid concentrate composition comprises about 1 to 10% w/w of the conditioning agent(s), for example, about 1% w/w, about 2% w/w, about 3% w/w, about 4% w/w, about 5% w/w, about 6% w/w, about 7% w/w, about 8% w/w, about 9% w/w or about 10% w/w.

In some embodiments the solid concentrate composition is in the form of powder, granule or tablet.

In some embodiments the solid concentrate composition is in the form of powder and/or granule.

In some embodiments, the liquid cleaning composition is a gel, e.g. a foaming gel, a hydrating gel or a cleaning gel.

In some embodiments, the liquid cleaning composition is a personal hygiene cleaning product, for example a hand soap, a toiletry soap, a hand cleaner, a face cleaner, a bodywash or a shampoo.

In some embodiments, the liquid cleaning composition is a household cleaning product, for example a general purpose cleaner, a household cleaner, a toilet cleaner, a dishwashing liquid, a bathroom cleaner, a kitchen cleaner, or a floor cleaner.

In some embodiments, the liquid cleaning composition comprises about 0.005 to 1% w/w of the fast-acting thickening agent.

In some embodiments, the liquid cleaning composition comprises about 0.01 to 0.5% w/w of the fast-acting thickening agent.

In some embodiments, the liquid cleaning composition comprises about 0.01 to 0.4% w/w of the fast-acting thickening agent.

In some embodiments, the liquid cleaning composition comprises about 0.02 to 0.3% w/w of the fast-acting thickening agent.

In some embodiments, the liquid cleaning composition comprises about 0.02 to 0.1% w/w of the fast-acting thickening agent.

In some embodiments, the liquid cleaning composition comprises about 0.05 to 4% w/w of the slow-acting thickening agent.

In some embodiments, the liquid cleaning composition comprises about 0.2 to 3% w/w of the slow-acting thickening agent.

In some embodiments, the liquid cleaning composition comprises about 0.3 to 2.5% w/w of the slow-acting thickening agent.

In some embodiments, the liquid cleaning composition comprises about 0.3 to. 1.5% w/w of the slow-acting thickening agent.

In some embodiments, the liquid cleaning composition is thixotropic.

In some embodiments, the liquid cleaning composition is a non-Newtonian fluid.

In some embodiments, the liquid cleaning composition has a pH of about 4 to 11.

In some embodiments, the liquid cleaning composition has a pH of about 9 to 10.

In some embodiments, the liquid cleaning composition has a pH of about 4.5 to 7. In some embodiments, the liquid cleaning composition has a pH of about 5 to 7. In some embodiments, the liquid cleaning composition has a pH of about 6 to 7. In some embodiments, the liquid cleaning composition has a pH of about 6.5 to 7.

In some embodiments the liquid cleaning composition has a viscosity of about 1,000 to 20,000 cP measured at 25° C. using a Brookfield LV type spindle viscometer at 12 RPM.

In some embodiments the liquid cleaning composition is suitable for pump action dispensing and has a viscosity of about 1,000 to 15,000 cP measured at 25° C. using a Brookfield LV type spindle viscometer at 12 RPM.

In some embodiments the liquid cleaning composition is suitable for dispensing from a bottle (for example a shampoo bottle) and has a viscosity of about 2,000 to 20,000 cP measured at 25° C. using a Brookfield LV type spindle viscometer at 12 RPM.

In some embodiments, an acceptable viscosity is achieved within about 1 hour, or within about 30 minutes, or within about 20 minutes, or within about 15 minutes, or within about 10 minutes, or within about 5 minutes of adding the solid concentrate composition to water or adding water to the solid concentrate composition.

In some embodiments, an acceptable viscosity is achieved in about 1 minute to 1 hour, or about 1 minute to 30 minutes, or about 1 minute to 20 minutes, or about 1 minute to 15 minutes, or about 1 minute to 10 minutes, or about 1 minute to 5 minutes of adding the solid concentrate composition to water, or adding water to the solid concentrate composition.

In some embodiments, the solid concentrate composition may achieve a substantially final or close to final viscosity in about 3 minutes to 2 hours, or about 3 minutes to 1 hour, or about 3 minutes to 30 minutes or about 3 minutes to 20 minutes of adding the solid concentrate composition to water, or adding water to the solid concentrate composition.

In some embodiments of the method of preparing a liquid cleaning composition or the method of cleaning a surface, the water has about 50 and 400 ppm calcium carbonate.

In some embodiments of the method of preparing a liquid cleaning composition or the method of cleaning a surface, the water is at a temperature of about 15° C. to 45° C.

In some embodiments of the method of preparing a liquid cleaning composition or the method of cleaning a surface, the solid concentrate composition may be added to about 100 to 10,000 mL of water, or about 100 to 5000 mL of water, about 200 to 3000 mL of water, or about 200 to 1000 mL of water, or about 100 to 10,000 mL of water, or about 100 to 5000 mL of water, about 200 to 3000 mL of water, or about 200 to 1000 mL of water added to the solid concentrate composition.

In some embodiments of the method of preparing a liquid cleaning composition or the method of cleaning a surface, the solid concentrate composition may be added to water, or water added to the solid concentrate composition, at about 0.5 to 20% w/v of the solid concentrate composition to water, or about 1 to 20% w/v, or about 5 to 20% w/v, or about 5 to 15% w/v, or about 5 to 10% w/v, or about 1 to 10% w/v, or about 1 to 6% w/v, or about 2 to 5% w/v, or about 2% w/v of the solid concentrate composition to water.

In some embodiments of the method of preparing a liquid cleaning composition or the method of cleaning a surface, the solid concentrate composition and water are combined without the aid of industrial mixers.

In some embodiments of the method of preparing a liquid cleaning composition or the method of cleaning a surface, the solid concentrate composition and water are combined without the aid of powered mixing.

In some embodiments of the method of preparing a liquid cleaning composition or the method of cleaning a surface, the solid concentrate composition and water are combined by shaking or agitating by hand.

In some embodiments of the method of preparing a liquid cleaning composition or the method of cleaning a surface, the solid concentrate composition and water are combined by shaking or agitating by hand with one or more mixing balls or an internal whisking device.

In some embodiments of the method of cleaning a surface, the solid concentrate composition is added to the water, or the water added to the solid concentrate composition at least about 1 minute prior to applying the liquid cleaning composition to the surface.

In some embodiments of the method of cleaning a surface, the solid concentrate composition is added to the water, or the water added to the solid concentrate composition at least about 2 minutes prior to applying the liquid cleaning composition to the surface.

In some embodiments of the method of cleaning a surface, the solid concentrate composition is added to the water, or the water added to the solid concentrate composition at least about 3 minutes prior to applying the liquid cleaning composition to the surface.

In some embodiments of the method of cleaning a surface, the solid concentrate composition is added to the water, or the water added to the solid concentrate composition at least about 5 minutes prior to applying the liquid cleaning composition to the surface.

In some embodiments of the method of preparing a solid concentrate composition, the method includes the step of granulating the solid concentrate composition.

In some embodiments of the method of preparing a solid concentrate composition, the method includes the step of dry mixing in a suitable blending mixer, for example a ribbon blender, paddle blender, or a Y or V blender.

In some embodiments, the blending mixer has a rotating container and the product is mixed by its dropping action.

In some embodiments of the method of preparing a solid concentrate composition, the method includes the step of pressing the solid concentrate powder through an extrusion device to produce noodle like granules.

In some embodiments of the method of preparing a solid concentrate composition, the method includes the step of forming a slurry of the thickening agents and surfactant by mixing with water and spray drying the slurry to produce a substantially homogeneous powder.

In some embodiments of the method of preparing a solid concentrate composition the method includes the step of tabletting the solid concentrate composition.

The term “comprising” as used in this specification and claims means “consisting at least in part of”. When interpreting each statement in this specification and claims that includes the term “comprising”, features other than that or those prefaced by the term may also be present. Related terms such as “comprise” and “comprises” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

As used herein the term “and/or” means “and” or “or”, or both.

As used herein “(s)” following a noun means the plural and/or singular forms of the noun.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

The disclosure consists in the foregoing and also envisages constructions of which the following gives examples only. Features disclosed herein may be combined into new embodiments of compatible components addressing the same or related inventive concepts.

### DETAILED DESCRIPTION

The inventors have discovered that a substantially uniform viscous liquid cleaning composition substantially free of lumps may be formed by mixing a solid concentrate composition with water. The solid concentrate composition contains a fast-acting thickening agent together with a slow-acting thickening agent (or slower hydrating agent) and at least one surfactant. The solid concentrate composition may allow for a viscous liquid cleaning composition to be prepared without the use of commercial mixers and at domestic sized quantity.

Described herein is a solid concentrate composition comprising a fast-acting thickening agent, a slow-acting thickening agent and a surfactant. The solid concentrate composition may be used for preparing a liquid cleaning composition. The liquid cleaning composition may be a viscous liquid cleaning composition.

Further or alternatively described herein is a method of preparing a liquid cleaning composition, the method comprising: a) providing a solid concentrate composition comprising: (i) a fast-acting thickening agent, (ii) a slow-acting thickening agent, and (iii) a surfactant; and b) adding the solid concentrate composition to water, or adding water to the solid concentrate composition, to provide the liquid cleaning composition.

Further or alternatively described herein is a liquid cleaning composition prepared according to the method described herein.

Further or alternatively described herein is a method of cleaning a surface, the method comprising: a) providing a solid concentrate composition comprising: (i) a fast-acting thickening agent, (ii) a slow-acting thickening agent, and (iii) a surfactant; and b) adding the solid concentrate composition to water, or adding water to the solid concentrate composition, to provide a liquid cleaning composition; and c) applying the liquid cleaning composition to the surface.

The solid concentrate composition described herein may be dilutable in water to provide a liquid cleaning composition. The solid concentrate composition may be diluted with water by the consumer. The dilution may be carried out under domestic conditions in relatively small batches (for example about 100 to 10,000 mL of water, about 100 to 5000 mL of water, about 200 to 3000 mL of water, or about 200 to 1000 mL of water) without the aid of industrial mixers. For example, the liquid cleaning composition may be shaken or stirred by hand by the consumer to aid dissolution and/or suspension of the solid concentrate composition.

The solid concentrate composition is formulated to provide a viscous liquid cleaning solution when diluted with water. The liquid cleaning solution may be suitable for use in a household dispensing device, such a pump action dispenser or a flip top squeeze bottle.

### Thickening Agents

Advantageously, the combination of fast-acting and slow-acting thickening agents described herein may reduce the formation of lumps and ‘fisheye’ formation. In some embodiments, there is no formation of lumps and/or ‘fisheye’ formation when the solid concentrate composition is added to water. Without wishing to be bound by theory, the inventors believe that the action of the fast-acting thickening agent may suspend the slow-acting thickening agent during the hydration process to reduce or prevent clumping of the slow-acting thickening agent.

The fast-acting thickening agent is an agent that hydrates quickly once mixed with water. Accordingly, the fast-acting thickening agent achieves a desired viscosity in a short amount of time. The fast-acting thickening agent may achieve at least about 80% of its total thickening action within about 2 minutes of addition to a liquid, or achieve at least about 80% of its total thickening action within about 1 minute of addition to a liquid. The fast-acting thickening agent may achieve at least about 90% of its total thickening action within about 2 minutes of addition to a liquid, or achieve at least about 90% of its total thickening action within about 1 minute of addition to a liquid.

The viscosity produced by the fast-acting thickening agent may be low enough allow the components (e.g. thickeners and surfactant) to be combined without powered mixing (for example with an industrial mixer) to allow mixing, or shaking or agitating by hand, but high enough to suspend the slow-acting thickening agent.

The fast-acting thickening agent may also be a thixotropic agent, for example an agent that makes a liquid thixotropic. The liquid cleaning composition may a non-Newtonian fluid or be thixotropic. This may depend on the choice of fast-acting thickening agent and/or slow-acting thickening agent. The fast-acting thickening agent may be a gelling agent or a thickening agent that is not gelling.

The solid concentrate composition may comprise about 0.1 to 5.0% w/w or about 0.1 to 4% w/w or about 0.1 to 3% w/w or about 0.1 to 2% w/w or about 0.2 to 2% w/w or about 0.3 to 2% w/w of the fast-acting thickening agent. The solid concentrate composition may comprise less than about 5% w/w, less than about 4% w/w, less than about 3% w/w, or less than about 2% w/w of the fast-acting thickening agent.

The fast-acting thickening agent may be selected from one or more of the group consisting of xanthan gum, guar gum, locust bean gum, carbomer, natural clays (such as attapulgite clay and montmorillonite clay), synthetic clays (such as Laponite). The fast-acting thickening agent may be xanthan gum.

The slow-acting thickening agent achieves a final viscosity over a longer amount of time than the fast-acting thickening agent. The slow-acting thickening agent may be an agent that hydrates slowly when combined with water.

The slow-acting thickening agent may achieve less than about 80% of its total thickening action 1 minute after addition to a liquid or achieve less than 80% of its total thickening action 2 minutes after addition to a liquid.

The slow-acting thickening agent may take at least about 10 minutes to hydrate once mixed with water. In some embodiments, the slow-acting thickening agent takes between about 10 minutes and 3 hours to hydrate once mixed with water.

The slow-acting thickening agent may achieve at least about 80% of its total thickening action within about 10 minutes to 3 hours of addition to a liquid or at least about 90% of its total thickening action within about 10 minutes to 3 hours of addition to a liquid.

The solid concentrate composition may comprise about 1 to 20% w/w, or about 2 to 18% w/w, or about 5 to 16% w/w, or about 8 to 15% w/w of the slow-acting thickening agent.

The slow-acting thickening agent may be is selected from one or more of the group consisting of carboxy methyl cellulose (CMC), hydroxy ethyl cellulose (HEC), methyl hydroxy ethyl cellulose (MHEC), methyl hydroxy propyl cellulose (MHPC), hydroxypropyl methyl cellulose (HPMC), modified starches, carrageenan and gellan. The slow-acting thickening agent may be HEC.

The slow-acting thickening agent may be less of a thixotropic agent than the fast-acting thickening agent. The slow-acting thickening agent may be a non-thixotropic agent.

Those persons skilled in the art will appreciate that the hydration rate and/or viscosity and/or final viscosity achieved by the fast-acting thickening agent and the slow-acting thickening agent may vary based on various factors, such as temperature, pH, and other components in the composition, e.g. electrolytes.

The person skilled in the art will be able to measure the total thickening action for the fast- and/or slow-acting thickener for any given concentration of thickener, fluid, temperature or other conditions, i.e. the viscosity a fluid stabilises at after addition of a thickener. A fluid will increase in viscosity after addition of a thickener, but the increase will stabilise over time (i.e. stop increasing) when the total thickening action is reached. The viscosity of a fluid a thickener has been added to may further change due to evaporation or decomposition or temperature after days, weeks or months, for example more than 1 week. This will be understood by a person skilled in the art and is not to be taken to be part of measurement of the total thickening action.

A person skilled in the art will also be able to measure the time taken for a thickening agent to reach total thickening action after addition to a liquid. For example, the viscosities and/or times taken to reach them discussed herein may be measured at 25° C. using a Brookfield LV type spindle viscometer at 12 RPM.

There may be more slow-acting thickening agent than fast-acting thickening agent in the solid concentrate composition and/or the liquid cleaning composition. The solid concentrate composition may comprise the fast-acting thickening agent and the slow-acting thickening agent in a ratio of about 1:5 to 1:40 by weight, or about 1:5 to 1:30 by weight, or about 1:5 to 1:25 by weight, or about 1:10 to 1:22 by weight. For example, for the ratio of about 1:5 to 1:40 by weight, a range between 1 part fast-acting thickening agent to 5 parts slow-acting thickening agent to 1 part fast-acting thickening agent to 40 parts slow-acting thickening agent by weight.

### Surfactant(s)

The solid concentrate composition comprises one or more surfactant(s), such as an anionic surfactant, a non-ionic surfactant, an amphoteric surfactant or a combination thereof. Those persons skilled in the art may select suitable surfactant(s) based on, for example, the intended use of the liquid cleaning composition. For example, suitable surfactants may be selected to improve the cleaning efficacy of the composition on specific soil types, modulate the viscosity of the composition, and modulate the foaming properties of the composition.

### Additional Components

The solid concentrate composition may further comprise one or more additives. Suitable additives include, but are not limited to, a pH modifying or stabilising agent(s), a chelating agent(s), an inert filler(s) or carrier agent(s), a fragrance(s) (preferably IFRA approved fragrances), a preservative(s), a humectant(s) and a conditioning agent(s) or a combination of any two or more thereof. Suitable additives are described herein and may be selected, for example, based on the intended use of the liquid cleaning composition. Additionally, those persons skilled in the art may will appreciate that other conventional additives known in the art may also be useful in the invention.

### Liquid Cleaning Composition

The solid concentrate composition may be added to water, or water added to the solid concentrate composition, to produce the liquid cleaning composition.

The combination of the fast-acting thickening agent and the slow-acting thickening agent allows for the liquid cleaning composition to be prepared at domestic scales (i.e. relatively small), For example, the solid concentrate composition may be added to about 100 to 10,000 mL of water, about 100 to 5000 mL of water, about 200 to 3000 mL of water, or about 200 to 1000 mL of water. Alternatively, about 100 to 10,000 mL of water, about 100 to 5000 mL of water, about 200 to 3000 mL of water, or about 200 to 1000 mL of water may be added to the solid concentrate composition. For example, the solid concentrate composition may be combined with water directly in a container the user can store and/or use to apply the liquid cleaning composition to a surface.

The solid concentrate composition may be added to water, or water added to the solid concentrate composition, at a rate of about 0.5 to 20% w/v of the solid concentrate composition to water, depending on the intended application, for example the solid concentrate composition may be added to water, or water added to the solid concentrate composition, at about 1 to 20% w/v, or about 1 to 10% w/v, or about 1 to 6% w/v, or about 2 to 5% w/v, or about 2% w/v of the solid concentrate composition to water. Alternatively, the solid concentrate composition may be added to water, or water added to the solid concentrate composition, at a rate of about 5 to 20% w/v or about 5 to 15% w/v, or about 5 to 10% w/v. The dilution rate may be varied between about 1 and 20% of solid concentrate product to water, or varied between about 5.0 and 20% of solid concentrate product to water.

The user may combine the solid concentrate composition and water without the aid of powered mixing, for example the user may shake or stir the mixture by hand. The shaking or agitating by hand may be aided with one or more mixing balls, or an internal whisking device, or other mixing aid.

The liquid cleaning composition may achieve an acceptable viscosity within about 2 hours, or within about 1 hour, or within about 30 minutes, or within about 20 minutes, or within about 15 minutes, or within about 5 minutes of adding the solid concentrate composition to the water, or adding the water to the solid concentrate composition. An acceptable viscosity is one which can be used by the consumer but may not be the final viscosity.

The liquid cleaning composition may achieve a substantially final or substantially stable or close to final or stable viscosity in about 3 minutes to 2 hours, or about 3 minutes to 1 hour, or about 3 minutes to 30 minutes or about 3 minutes to 20 minutes of adding the solid concentrate composition to water, or adding water to the solid concentrate composition. The “final viscosity” or “stable viscosity” should be taken to mean the viscosity the liquid cleaning composition stabilises at after the initial increase in viscosity after combining the concentrated solid composition with water. The viscosity of liquid cleaning composition may further change from the final or stable viscosity due to evaporation or decomposition or temperature change after days, weeks or months, for example more than 1 week. This will be understood by a person skilled in the art.

The desired viscosity of the liquid cleaning composition will depend on the intended application. For example, a liquid cleaning composition to be used in a pump action bottle, e.g. a hand cleaner, may have a viscosity of about 1,000 to 20,000 cP measured at 25° C. using a Brookfield LV type spindle viscometer at 12 RPM, or about 1,000 to 15,000 cP measured at 25° C. using a Brookfield LV type spindle viscometer at 12 RPM. In another example, a liquid cleaning composition to be used as a shampoo dispensed from a bottle may have a viscosity of about 2,000 to 20,000 cP measured at 25° C. using a Brookfield LV type spindle viscometer at 12 RPM.

The liquid cleaning composition may be a non-Newtonian fluid. The liquid cleaning composition may be thixotropic.

The solid concentrate composition is suitable for providing the liquid cleaning composition under acidic, alkaline or neutral conditions. For example, the liquid cleaning composition may have a pH of about 4 to 11.

The solid concentrate composition may be suitable for providing the liquid cleaning composition under alkaline conditions. For example, the liquid cleaning composition may have a pH of about 9 to 10, such as when the liquid cleaning composition is a toiletry soap.

The solid concentrate composition may alternatively be suitable for providing the liquid cleaning composition under acidic or neutral conditions. For example, the liquid cleaning composition may have a pH of about 6 to 7, such as when the liquid cleaning composition is a shampoo. Advantageously, a desirable thickening effect may be achieved at a pH of about 6.5 to 7.

In some embodiments, the solid concentrate composition is suitable for providing the liquid cleaning composition under acidic conditions. For example, the liquid cleaning composition may have a pH of about 5 to 6.

The solid concentrate composition is suitable for providing the liquid cleaning composition when diluted with various types of water typically found in most homes, such as soft or moderately hard water. For example, with about 50 to 400 ppm calcium carbonate.

Various types of household and personal hygiene cleaning products may be prepared from the solid concentrate composition. In some embodiments, the liquid cleaning composition is suitable for use as a personal hygiene cleaning product, such as a hand soap, a bodywash or a shampoo. In some embodiments, the liquid cleaning composition is suitable for use as a household cleaning product, such as an all-purpose cleaner, a toilet cleaner, a kitchen cleaner or a bathroom cleaner. The solid concentrate composition may be formulated to provide the liquid cleaning composition suitable for such uses by simply adding the solid concentrate composition to water, or adding water to the solid concentrate composition.

Those persons skilled in the art will understand that the final viscosity of the liquid cleaning composition may depend on several factors, such as pH of the liquid cleaning composition, the amount of the thickening agents, and the amount of other components, such as surfactants and electrolytes.

### Method of Preparing a Solid Concentrate Composition

The solid concentrate composition can be prepared by various methods depending on the final format desired, such as powder, granules or tablets. For example, the method of preparing a solid concentrate composition may include the step of granulating the solid concentrate composition, forming a powder, or tabletting the solid concentrate composition.

For example, for a powder or granule (e.g. to be filled into small containers or sachets), the formulations may be dry mixed in a suitable blending mixer, such as a ribbon blender, paddle blender, or a Y or V blender etc. Preferred blending mixers have a rotating container and the product is mixed by its dropping action. Low shear blending methods may be used. Under suitable conditions granulation can be achieved which can be used to produce a substantially free flowing product that aids packing.

Alternatively, the powder may be pressed through an extrusion device to produce suitable noodle like granules (for example, a reciprocating screen type granulator).

Tablets may be pressed in tableting machine, for example such as those used for pharmaceuticals or dishwashing tablet manufacture. However, tablets are generally less preferred, due to the desire to control the speed of the thickening agent action.

A substantially homogeneous powder may be produced by forming a slurry of the mixed components with water and spray drying. Fragrance may be blended in after powder formation to avoid losses through evaporation and reduce flammability risks while drying. However, spray drying may be less preferred as present methods of spray drying are both capital and energy intensive and result in a more bulky product that requires larger packaging volume. The carbon footprint of the product is increased and therefore is less desirable where the intension to minimise environmental impact.

Where, in the foregoing description reference has been made to integers or components having known equivalents thereof, those integers are herein incorporated as if individually set forth.

Although the present disclosure has been described in terms of certain embodiments, other embodiments apparent to those of ordinary skill in the art also are within the scope of this disclosure. Thus, various changes and modifications may be made without departing from the spirit and scope of the disclosure. For instance, various components may be repositioned as desired. Moreover, not all of the features, aspects and advantages are necessarily required to practice the present disclosure. Accordingly, the scope of the present disclosure is intended to be defined only by the claims that follow.

### Examples

### 1. Examples of Viscous Hand Cleaning Products

The following Example provides examples of viscous hand cleaning products that are suitable for pump action dispensers and flip top squeeze bottles.

### 1.1 Examples of Solid Concentrate Compositions for Use to Prepare Viscous Hand Cleaning Products

TABLE 1

| | Amount % w/w | | | |
|---|---|---|---|---|
| | HC | HC | HC | |
| Ingredient | example 1 | example 2 | example 3 | Function |
| Alpha olefin | 35.0 | 40.0 | 20.0 | Anionic |
| sulfonate | | | | surfactant |
| Sodium | 0.0 | 0.0 | 20.0 | Anionic |
| Cocosulfate | | | | surfactant |
| Sodium | 10.0 | 10.0 | 10.0 | Anionic |
| Lauroyl | | | | surfactant |
| Glutamate | | | | |
| Alkyl | 5.0 | 0.0 | 0.0 | Non-ionic |
| polyglucoside | | | | surfactant |
| C8/C10 | | | | |
| Citric acid | 1.0 | 1.0 | 1.0 | Acidifier |
| Sodium | 4.0 | 4.0 | 4.0 | Natural |
| Citrate | | | | Chelating |
| | | | | agent |
| HEC | 11.0 | 11.0 | 11.0 | Thickener |
| | | | | (slow |
| | | | | acting) |
| Xanthan gum | 0.5 | 0.5 | 0.5 | Thickener |
| | | | | (fast |
| | | | | acting) |
| Betain | 5.0 | 5.0 | 5.0 | Humectant |
| Fragrance | 2.0 | 2.0 | 2.0 | |
| Sodium | 0.5 | 0.5 | 0.5 | Preservative |
| Benzoate | | | | |
| Sodium | 10.0 | 10.0 | 10.0 | Inert filler |
| sulfate | | | | carrier |
| Maltodextrin | 16.0 | 16.0 | 16.0 | Inert Filler |
| | | | | carrier |
| Total | 100.0 | 100.0 | 100.0 | |

The compositions in the Table 1 may be diluted with soft or even moderately hard water typically found in most homes. The dilution rate can be varied between 5.0 and 20% of product to water depending on the desired viscosity and foaming/cleaning power. Small granules and powder will be fully dissolved or suspended within 5 minutes of mixing with lukewarm water. Tablets may be less suitable for thickened products such as these.

### 2. Examples of Viscous Shampoo and Body Wash Products

The following examples demonstrate the versatility of the fast-acting/slow-acting thickening agent combination for thickening cleansing products. The products are suitable for pump action dispensers or flip top squeeze bottle. This example provides compositions with a different surfactant combination, but which still achieves the thickening effect. Different surfactants may be used to provide desired properties, such as foaming and mildness.

### 2.1 Examples of Solid Concentrate Compositions for Use to Prepare Viscous Shampoo and Body Wash Products

Table 2 shows examples of solid concentrate compositions that may be diluted with water to provide viscous shampoo and body wash products.

TABLE 2

| | Amount % w/w | | | |
|---|---|---|---|---|
| | Shampoo | Shampoo | Body wash | |
| Ingredient | example 1 | example 2 | example 1 | Function |
| Alpha olefin | 0.0 | 20.0 | 20.0 | Anionic |
| sulfate | | | | surfactant |
| Sodium Lauroyl | 50.0 | 30.0 | 30.0 | Anionic |
| Glutamate | | | | surfactant |
| Alkyl | 3.0 | 3.0 | 3.0 | Non-ionic |
| polyglucoside | | | | surfactant |
| C₁₀-C₁₄ | | | | |
| Citric acid | 0.5 | 0.5 | 0.5 | Acidifier |
| Sodium Citrate | 2.0 | 2.0 | 2.0 | Natural |
| | | | | Chelating |
| | | | | agent |
| HEC | 10.0 | 10.0 | 10.0 | Thickener |
| | | | | (slow acting) |
| Xanthan gum | 1.0 | 1.0 | 1.0 | Thickener |
| | | | | (fast acting) |
| Betain | 5.0 | 5.0 | 5.0 | Humectant |
| Fragrance | 2.0 | 2.0 | 2.0 | |
| Potassium sorbate | 0.5 | 0.5 | 0.5 | Preservative |
| Maltodextrin | 18.0 | 18.0 | 24.0 | Inert Filler |
| | | | | carrier |
| Hydroxypropyl | 3.0 | 3.0 | 2.0 | Conditioning |
| guar gum | | | | agent |
| Polyquaternium 10 | 5.0 | 5.0 | 0.0 | Conditioning |
| | | | | agent |
| Total | 100.0 | 100.0 | 100.0 | |

2.2 Dilution with Water to Produce Ready to Use Viscous Shampoo and Body Wash Products

The compositions in the Table 2 above may be diluted with soft or even moderately hard water typically found in most homes. The dilution rate can be varied between 5.0 and 20% of product to water depending on the desired viscosity and foaming/cleaning power. Small granules and powder will be fully dissolved or suspended within 5 minutes of mixing with lukewarm water and have a pH of 5 to 7.

1. A solid concentrate composition comprising: a fast-acting thickening agent, a slow-acting thickening agent, and a surfactant.

2. A method of preparing a liquid cleaning composition, the method comprising:
a) providing a solid concentrate composition comprising:
(i) a fast-acting thickening agent,
(ii) a slow-acting thickening agent, and
(iii) a surfactant; and
b) adding the solid concentrate composition to water, or adding water to the solid concentrate composition, to provide the liquid cleaning composition.

3. A method of cleaning a surface, the method comprising:
a) providing a solid concentrate composition comprising:
(i) a fast-acting thickening agent,
(ii) a slow-acting thickening agent, and
(iii) a surfactant; and
b) adding the solid concentrate composition to water, or adding water to the solid concentrate composition, to provide a liquid cleaning composition; and
c) applying the liquid cleaning composition to the surface.

4. A method of preparing a solid concentrate composition, the method comprising combining:
a) a fast-acting thickening agent,
b) a slow-acting thickening agent, and
c) a surfactant.

5. The composition of claim 1 or the method of any one of claims 2 to 4, wherein the solid concentrate composition comprises about 0.1 to 5.0% w/w of the fast-acting thickening agent.

6. The composition of claim 1 or 5 or the method of any one of claims 2 to 5, wherein the solid concentrate composition comprises, less than about 5% w/w, less than about 4% w/w, less than about 3% w/w, less than about 2% w/w, or less than about 1% w/w of the fast-acting thickening agent.

7. The composition of any one of claims 1, 5 and 6 or the method of any one of claims 2 to 6, wherein the fast-acting thickening agent provides at least about 80% of its total thickening action within about 2 minutes of addition to a liquid.

8. The composition of any one of claims 1 and 5 to 7 or the method of any one of claims 2 to 7, wherein the fast-acting thickening agent is a gelling agent.

9. The composition of any one of claims 1 and 5 to 8 or the method of any one of claims 2 to 8, wherein the fast-acting thickening agent is a thixotropic agent.

10. The composition of any one of claims 1 and 5 to 9 or the method of any one of claims 2 to 9, wherein the fast-acting thickening agent is selected from the group consisting of xanthan gum, guar gum, locust bean gum, carbomer, natural clays (such as attapulgite clay and montmorillonite clay), synthetic clays (such as Laponite) and a combination of any two or more thereof.

11. The composition of any one of claims 1 and 5 to 10 or the method of any one of claims 2 to 10, wherein the solid concentrate composition comprises about 1 to 20% w/w of the slow-acting thickening agent.

12. The composition of any one of claims 1 and 5 to 11 or the method of any one of claims 2 to 11, wherein the slow-acting thickening agent provides less than about 80% of its total thickening action about 1 minute after addition to a liquid.

13. The composition of any one of claims 1 and 5 to 12 or the method of any one of claims 2 to 12, wherein the slow-acting thickening agent provides at least about 80% of its total thickening action within about 10 minutes to 3 hours of addition to a liquid.

14. The composition of any one of claims 1 and 5 to 13 or the method of any one of claims 2 to 13, wherein the slow-acting thickening agent is selected from the group consisting of carboxy methyl cellulose (CMC), hydroxy ethyl cellulose (HEC), methyl hydroxy ethyl cellulose (MHEC), methyl hydroxy propyl cellulose (MHPC), hydroxypropyl methyl cellulose (HPMC), modified starches, carrageenan, gellan and a combination of any two or more thereof.

15. The composition of any one of claims 1 and 5 to 14 or the method of any one of claims 2 to 14, wherein the slow-acting thickening agent is a less thixotropic agent than the fast-acting thickening agent.

16. The composition of any one of claims 1 and 5 to 14 or the method of any one of claims 2 to 15, wherein the solid concentrate composition comprises the fast-acting thickening agent and the slow-acting thickening agent in a ratio of about 1:5 to 1:40 by weight.

17. The composition of any one of claims 1 and 5 to 16 or the method of any one of claims 2 to 16, wherein the surfactant is selected from the group consisting of an anionic surfactant, an amphoteric surfactant, a non-ionic surfactant and a combination of any two or more thereof.

18. The composition or the method of claim 17, wherein the anionic surfactant is selected from the group consisting of a secondary alkyl sulfonate, an olefin sulfonate, a soap of fatty acids, an alkylbenzene sulfonate, an alcohol sulfate, an alcohol ether sulfate, an alkyl sulfate, an alkyl sulphosuccinate, a sulfosuccinic derivative, an alkyl ether carboxylate, an alkyl phosphate, an alkylether phosphate, a fatty acid isethionate, an acyl sarcosinate, a fatty acid/amino acid ester, a fatty alcohol amino acid ester, a fatty alcohol amino acid ester adduct, an amino acid derivative, a salt thereof and a combination of any two or more thereof.

19. The composition or the method of claim 17, wherein the non-ionic surfactant is selected from the group consisting of an ethoxylated fatty alcohol, an ethoxylated nonyl phenol, an ethoxylated fatty acid, a fatty acid alanolamide, an ethoxylated fatty amine, a fatty amine oxides glyceryl fatty ester, a sorbitan, an ethoxylated sorbitan ester, a sucrose ester, an alkyl polyglucoside, an ethylene/propylene oxide block copolymer, an ethoxylated fatty alcohol, a propoxylated fatty alcohol and a combination of any two or more thereof.

20. The composition or the method of claim 17, the amphoteric surfactant is selected from the group consisting of an alkyl dimethyl betaine, an alkyl amido betaine, an alkyl sulphobetaine, an alkyl amido sulphobetaine, an imidazoline betaine derivative, an alkyl amino propionate and a combination of any two or more thereof.

21. The composition or the method of claim 17, wherein the surfactant is a sodium or potassium salt.

22. The composition of any one of claims 1 and 5 to 21 or the method of any one of claims 2 to 21, wherein the solid concentrate composition comprises about 30 to 70% w/w of the surfactant.

23. The composition of any one of claims 1 and 5 to 22 or the method of any one of claims 2 to 22, wherein the solid concentrate composition comprises one or more of a pH modifying or stabilising agent(s), a chelating agent(s), an inert filler(s) or carrier agent(s), a fragrance(s), a preservative(s), a humectant(s), a conditioning agent(s) and a combination of any two or more thereof.

24. The composition of any one of claims 1 and 5 to 23 or the method of any one of claims 2 to 23, wherein the solid concentrate composition is in the form of a powder, granule or tablet.

25. A liquid cleaning composition prepared according to the method of claim 2 or any one of claims 5 to 25 when dependent on claim 2.

26. The liquid cleaning composition of claim 25 or the method of claim 2 or 3 or any one of claims 5 to 25 when dependent on claim 2 or 3, wherein the liquid cleaning composition is a gel.

27. The liquid cleaning composition of claim 25 or 26 or the method of claim 2 or 3 or any one of claims 5 to 24 when dependent on claim 2 or 3, wherein the liquid cleaning composition is a personal hygiene cleaning product or a household cleaning product.

28. The liquid cleaning composition of any one of claims 25 to 27 or the method of claim 2 or 3 or any one of claims 5 to 24 when dependent on claim 2 or 3, wherein the liquid cleaning composition comprises about 0.005 to 1% w/w of the fast-acting thickening agent.

29. The liquid cleaning composition of any one of claims 25 to 28 or the method of claim 2 or 3 or any one of claims 5 to 24 when dependent on claim 2 or 3, wherein the liquid cleaning composition comprises about 0.05 to 4% w/w of the slow-acting thickening agent.

30. The liquid cleaning composition of any one of claims 25 to 29 or the method of claim 2 or 3 or any one of claims 5 to 24 when dependent on claim 2 or 3, wherein the liquid cleaning composition has a pH of about 4 to 11 or about 9 to 10.

31. The liquid cleaning composition of any one of claims 25 to 30 or the method of claim 2 or 3 or any one of claims 5 to 24 when dependent on claim 2 or 3, wherein the liquid cleaning composition has a viscosity of about 1,000 to 20,000 cP measured at 25° C. using a Brookfield LV type spindle viscometer at 12 RPM.

32. The liquid cleaning composition of any one of claims 25 to 31 or the method of claim 2 or 3 or any one of claims 5 to 24 when dependent on claim 2 or 3, wherein an acceptable viscosity is achieved within about 1 hour, or within about 30 minutes, or within about 20 minutes, or within about 15 minutes, or within about 10 minutes, or within about 5 minutes of adding the solid concentrate composition to the water, or adding the water to the solid concentrate composition.

33. The liquid cleaning composition of any one of claims 25 to 31 or the method of claim 2 or 3 or any one of claims 5 to 24 when dependent on claim 2 or 3, wherein an acceptable viscosity is achieved in about 1 minute to 1 hour, or about 1 minute to 30 minutes, or about 1 minute to 20 minutes, or about 1 minute to 15 minutes, or about 1 minute to 10 minutes, or about 1 minute to 5 minutes of adding the solid concentrate composition to water, or adding water to the solid concentrate composition.

34. The method of claim 2 or 3 or any one of claims 5 to 24 when dependent on claim 2 or 3, wherein the water is at a temperature of about 15° C. to 45° C.

35. The method of claim 2 or 3 or any one of claims 5 to 24 when dependent on claim 2 or 3, wherein the solid concentrate composition is added to about 100 to 10,000 mL of water, or about 100 to 5000 mL of water, or about 200 to 3000 mL of water, or about 200 to 1000 mL of water, or about 100 to 10,000 mL of water, or about 100 to 5000 mL of water, or about 200 to 3000 mL of water, or about 200 to 1000 mL of water is added to the solid concentrate composition.

36. The method of claim 2 or 3 or any one of claims 5 to 24 when dependent on claim 2 or 3, wherein the solid concentrate composition is added to water, or water is added to the solid concentrate composition, at about 0.5 to 20% w/v of the solid concentrate composition to water, or about 1 to 20% w/v, or about 5 to 20% w/v, or about 5 to 15% w/v, or about 5 to 10% w/v, or about 1 to 10% w/v, or about 1 to 6% w/v, or about 2 to 5% w/v, or about 2% w/v of the solid concentrate composition to water.

37. The method of claim 2 or 3 or any one of claims 5 to 24 when dependent on claim 2 or 3, further comprising combining the solid concentrate composition and water by shaking or agitating by hand.

38. The method of claim 4 or any one of claims 5 to 24 when dependent on claim 4, wherein the method includes the step of granulating the solid concentrate composition.

39. The method of claim 4 or any one of claims 5 to 24 when dependent on claim 4, wherein the method includes the step of dry mixing in a suitable blending mixer, for example a ribbon blender, paddle blender, or a Y or V blender.

40. The method of claim 4 or any one of claims 5 to 24 when dependent on claim 4, wherein the method includes the step of forming a slurry of the thickening agents and surfactant by mixing with water and spray drying the slurry to produce a substantially homogeneous powder.

41. The method of claim 4 or any one of claims 5 to 24 when dependent on claim 4, wherein the method includes the step of tabletting the solid concentrate composition.
